(19) United States
(12) Plant Patent Application Publication
(10) Pub. No.: US 2017/0231141 P1
(43) Pub. Date: Aug. 10, 2017

(54) Hlbiscus 'ILVOPS'

(50) ;

(71) Applicant: Johan van Huylenbroeck, Melle (BE)

(72) Inventor: Johan van Huylenbroeck, Melle (BE)

(21) Appl. No.: 14/998,771

(22) Filed: Feb. 10, 2016

(51) Int. Cl. A01H 5/02

(52) U.S. Cl. PLT/257

(57) ABSTRACT

A new and distinct Hibiscus cultivar named ‘ILVOPS’ is disclosed, characterized by and heavy blooming of large purple single flowers with a red-purple center eye. Plants do not produce seeds. The new variety is a Hibiscus, normally produced as an outdoor garden or container plant.

Latin name of the genus and species: Hibiscus syriacus

Variety denomination: ‘ILVOPS’

### BACKGROUND OF THE INVENTION

The new Hibiscus cultivar is a product of a planned breeding program conducted by the inventor, Johan van Huylenbroek, in Melle, Belgium. The objective of the breeding program was to produce new sterile triploid Hibiscus varieties. The cross resulting in this new variety was made during 1998.

The seed parent is the unpatented variety Hibiscus syriacus ‘Purple CV2’ (a tetraploid cultivar that was obtained from South Korea). The pollen parent is the patented, commercial variety Hibiscus syriacus U.S. Plant Pat. No. 12,196. The new variety was identified as a potentially interesting selection in July of 2001, at a commercial greenhouse in Melle, Belgium.

Asexual reproduction of the new cultivar ‘ILVOPS’ by vegetative soft wood cuttings was first performed during 2001, at a commercial greenhouse in Melle, Belgium. Subsequent propagation has shown that the unique features of this cultivar are stable and reproduced true to type in 6 successive generations.

### SUMMARY OF THE INVENTION

The cultivar ‘ILVOPS’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘ILVOPS’ These characteristics in combination distinguish ‘ILVOPS’ as a new and distinct Hibiscus cultivar:
1. Sterile variety, producing no seed.
2. Large, purple single flowers with a red-purple center eye.
3. High flower production per plant.

### PARENT COMPARISON

Plants of the new cultivar ‘ILVOPS’ are similar to plants of the seed parent variety Hibiscus syriacus ‘Purple CV2’ in most horticultural characteristics, however, plants of the new cultivar ‘ILVOPS’ differ in the following;
1. Smaller flowers than the seed parent.

Plants of the new cultivar ‘ILVOPS’ are similar to plants of the pollen parent variety Hibiscus syriacus ‘Floru’ U.S. Plant Pat. No. 12,196 in most horticultural characteristics, however, plants of the new cultivar ‘ILVOPS’ differ in the following;
1. Larger flowers.
2. Red-purple eye, whereas the pollen parent has a red eye.
3. Purple/pink flowers, whereas the pollen parent has more vibrant pink/red flowers. COMMERCIAL COMPARISON

Plants of the new cultivar ‘ILVOPS’ can be compared to the variety Hibiscus ‘ILVO347’ PPAF. These varieties are similar in most horticultural characteristics; however ‘ILVOPS’ differs in the following:
1. Large purple flowers with red-purple centers. The comparator has large pink flower with red centers.

### BRIEF DESCRIPTION OF THE PHOTOGRAPHS

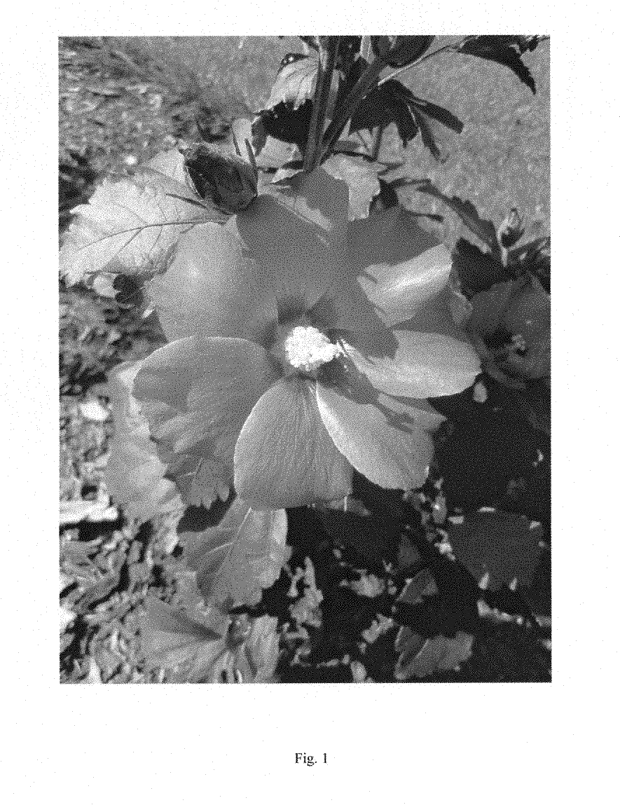

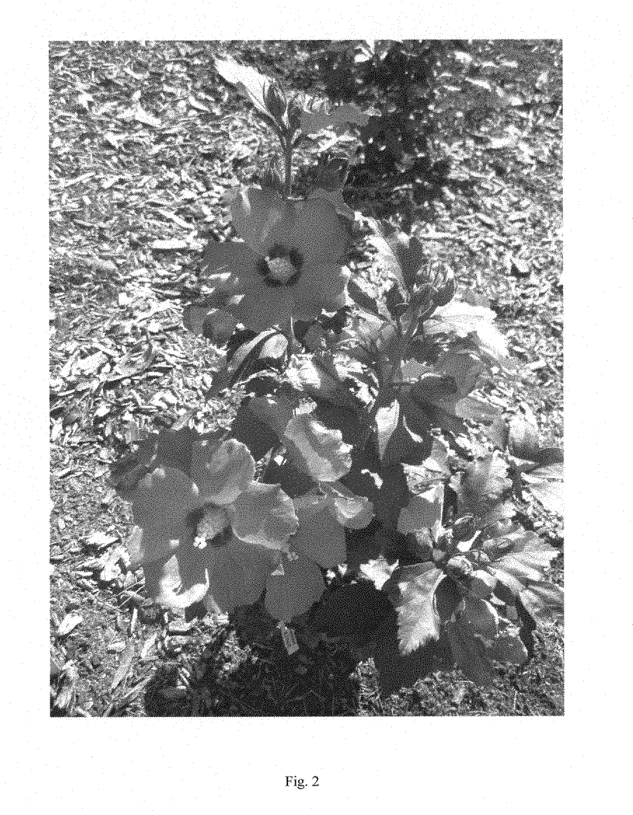

The accompanying photograph in FIG. 1 illustrates in full color a typical flower of of ‘ILVOPS’ grown in an outdoor flower bed, in Grand Haven, Mich. FIG. 2 illustrates in full color a typical plant of ‘ILVOPS’. Age of the plant photographed is approximately 1.5 years. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 1996 except where general terms of ordinary dictionary significance are used. The following observations and measurements describe ‘ILVOPS’ plants grown in a commercial poly greenhouse in Grand Haven, Mich. under natural lighting. Measurements were taken during the July of 2015. The plants were 2 to 3 years old in 2 gallon containers. The growing temperature ranged from 5° C. to 27° C. Measurements and numerical values represent averages of typical plant types.
Botanical classification: Hibiscus syriacus ‘ILVOPS’
Propagation:
   Time to initiate roots.—20 days at approximately 25° C.
   Root description.—Fibrous, medium root thickness. Tan/brown in color, not effectively measured with a color chart.
   Time to produce a rooted young plant.—90 days at 25° C.
Plant:
   Plant type.—Perennial shrub.
   Age of plant described.—Approximately 2-3 years old.
   Growth habit.—Upright.
   Pot size of plant described.—3 gallon pot.
   Overall plant shape.—Upright.
   Height.—67 cm.
   Plant spread.—72 cm.
   Growth rate.—Moderate.
   Plant vigor.—Good.
   Branching characteristics.—Basal branching. Some pinching required to encourage a denser plant habit.
   Length of primary lateral branches.—26.5 cm.
   Diameter of lateral branches.—4 mm.
   Quantity of lateral branches.—8-10.
   Stem.—Stem shape: Round Stem strength: Moderate Color: Stem: Near RHS Green 141A Bark: Near RHS Grey-brown 199B Pubescence: Slightly. Angle of attachment: 25-50° from center of plant.
   Internode length.—9 cm on average.
Foliage:
   Leaf.—Arrangement: Sub-opposite to alternate, single Leaf shape: Ovate Average Length: 7.3 cm Average Width: 4.7 cm Apex: Acute Base: Cuneate Margin: Lobed Texture of top surface: Smooth, waxy Texture of bottom surface: Smooth, waxy Leaf pubescence: None. Color: Young foliage upper side: Near RHS Green 137A Young foliage under side: Near RHS Yellow-green 144A Mature foliage upper side: Near RHS Yellow-green 147A Mature foliage under side: Near RHS Yellow-green 147B Venation: Pattern: Palmate Color upper side: Near RHS Yellow-green 147A Culur under side: Near RHS Yellow-green 147A Petiole: Average Length: 2 cm Diameter: 2 mm Petiole color upper side: Near RHS Green 137B Petiole color lower side: Near RHS Green 137B Petiole Texture upper side: Smooth; pubescent.
Flower:
   Natural flowering season.—Early June through Summer.
   Flowering form.—Single rotate.
   Flower habit.—Terminal.
   Flower longevity on plant.—About 2-3 days.
   Flower longevity as a cut flower.—About 1 day.
   Quantity of flowers per plant.—About 100.
   Flowers per lateral branch.—8 to 12.
   Flower shape.—Rotate.
   Rate of flower opening.—Fast/Moderate.
   Flower habit.—Single.
   Flower aspect.—Outward/upright.
   Size.—Length: 5 cm Diameter: 5 cm Depth: 5 cm.
   Flower other characteristics.—Persistence: Self-cleaning. Fragrance: None.
   Bud.—Shape: Rounded cone Length: Immature: 2 cm. Mature 3.5 cm. Diameter: Immature: 1.5 mm. Mature 2.5 cm. Color: Immature: Near RHS Violet-blue 91D. Mature: Near RHS Purple 78A.
   Petal.—Petal arrangement: Whorled Number of petals per flower: 6 Fused: No Petal Shupe: Obovate Petal Base: Truncate Margin: Entire Tip shape: Emerginate and spurred Length: 6 cm Width: 5.5 cm Texture: Upper: Smooth/leather; waxy Lower: Waxy; with prominent smooth veins.
   Petal Color.—When opening: Upper surface: Near RHS Purple-violet 80B; Red-purple 59A near base of petal. Lower surface: Near RHS Purple 78B Fully opened: Upper surface: Near RHS Purple 78B; Red-purple 59A near base of petal. Lower surface: Near RHS Purple 78B Fading to: Upper surface: Near RHS Purple 78A with veins and margin fading to Purple 79A Lower surface: Near RHS Purple 78A with veins and margin fading to Purple 79A.
   Sepal.—Arrangement: Radial; forming a star shaped calyx Number: 6 Shape: Ovate Ari state Base: Fused Margin: Entire Length: 3 cm Width: 1 cm Texture, Upper: Rough; slightly pubescent. Texture, Lower: Smooth; waxy Color: Upper surface at maturity: Calyx: Near RHS Yellow-green 144A; Epicalyx: Near RHS Green 137A Under surface at maturity: Calyx: Near RHS Yellow-green 145A; Epicalyx: Near RHS Green 137A.
   Calyx.—Shape: Calyx: Ovate; star. Epicalyx: Acicular Length: Calyx: 3 cm. Epicalyx: 3 cm Diameter: Calyx: 1 cm. Epicalyx: 2 cm.
   Peduncle.—Length: 4 cm Diameter: 5 min Color: Near RHS Yellow-Green 146B Orientation: 25° Strength: Moderate Texture: Rough/waxy; pubescent.
Reproductive organs:
   Stamens.—Number: About 45 Filament length: 3 mm Filament color: Near RHS White 155B.
   Anthers.—Shape: Oval lobed Length: 4 mm Color: Near RHS Greyed-yellow 160C Pollen color: Near RHS White 155B Pollen amount: Some.
   Pistil.—Number: 5-6 lobed Length: 0.5-1 cm Style: Length: 3 cm Color: Near RHS White 155B Stigma: Shape: Round Color: Near RHS White 155A Ovary color: Near RHS White 155B.
Other characteristics
   Seeds and fruits.—Not observed.
   Disease/pest resistance.—Neither resistance nor susceptibility to normal diseases and pests of Hibiscus have been observed.
   Garden performance.—Good tolerance, performs well in harsh weather.
   Temperature tolerance.—The new variety tolerates temperatures between −26° C. to 35° C.

What is claimed is:

1. A new and distinct cultivar of Hibiscus plant named ‘ILVOPS’ as herein illustrated and described.
